(19) United States
(12) Patent Application Publication
(10) Pub. No.: US 2007/0037709 A1
(43) Pub. Date: Feb. 15, 2007

(54) Polyethylene glycol esters of r-(+)-mcpp acid

(76) Inventors: Hermann Uhr, Leverkusen (DE); Rainer Bruns, Leverkusen (DE); Heinz-Joachim Rother, Krefeld (DE); Gerd-Friedrich Renner, Kurten (DE)

Correspondence Address: Lanxess Corporation;Patent Department, 111 Ridc Park West Drive, Pittsburgh, PA, 15275-1112, US

(21) Appl. No.: 10/554,322

(22) PCT Filed: Apr. 27, 2004
(86) PCT No.: PCT/EP04/04414

§ 371 Date: Sep. 5, 2006

(30) Foreign Application Priority Data
May 7, 2003 (DE) 10320551.9

(51) Int. Cl. A01N 39/02; A01N 25/16; C07C 69/76

(52) U.S. Cl. 504/317; 504/363; 560/63

(57) ABSTRACT

The novel enantiomerically enriched R-(+)-2-(4-chloro-2-methylphenoxy)-propionic acid polyethylene glycol esters and any of their mixtures with one another are outstandingly suitable as root penetration inhibitors and can be employed as such for the protection of buildings and building materials.

The present invention relates to novel enantiomerically enriched R-(+)-2-(4-chloro-2-methylphenoxy)propionic acid polyethylene glycol esters, any of their mixtures with one another, processes for their preparation, and their use as root penetration inhibitors.

The penetration of roots through building materials results in undesirable damage to the materials. Plant roots are capable of destroying, in particular, plastically deformable materials such as sealants, roofing membranes, but also bitumen. The penetration of roots into seals of sewers and waste-water pipes, into the covering of flat roofs, into bitumen insulations of pipelines, bridge constructions and hydraulic structures, and the penetration of roots into and through light bitumen roads are generally known problems. Leaks, corrosion, and damage to buildings, roads and pipelines may result.

To avoid this type of damage, for example F. Hegemann, Abiogene Bitumenadditive, Bitumen-Teere-Asphalte-Peche 24, 105 (1973) describes the addition of root-inhibitory active ingredients to building materials.

DE-A 1196115 discloses polyethylene glycol esters of 2-(4-chloro-2-methylphenoxy)propionic acid which have a root-inhibitory effect.

DE-A 4412330 describes processes for the production of polyethylene glycol esters of 2-(4-chloro-2-methylphenoxy)propionic acid which start from polyethylene glycols with a molar mass distribution of 170 to 230, and the resulting products are said to have improved root-inhibitory properties.

WO 9506408 describes the use of specific esters of 2-(4-chloro-2-methylphenoxy)propionic acid for the protection of buildings, building materials and insulation compounds against root penetration.

Moreover, it is known that, in herbicidal applications in crop protection where 2-(4-chloro-2-methylphenoxy)propionic acid is employed, the R-(+) isomer is markedly more effective than the S-(−) isomer or the racemic mixture (cf. B. Åberg, Swedish J. agric. Res. 1973, 3, 49).

Surprisingly, novel, enantiomerically enriched polyethylene glycol esters of R-(+)-2-(4-chloro-2-methylphenoxy)propionic acid of the general formula (I) and their mixtures with one another which have a markedly better activity as root inhibitors than corresponding polyethylene glycol esters of racemic 2-(4-chloro-2-methylphenoxy)propionic acid or polyethylene glycol esters of S-(−)-2-(4-chloro-2-methylphenoxy)propionic acid have now been found.

The present invention relates to enantiomerically enriched polyethylene glycol esters of R-(+)-2-(4-chloro-2-methylphenoxy)propionic acid of the general formula (I) and any of their mixtures with one another 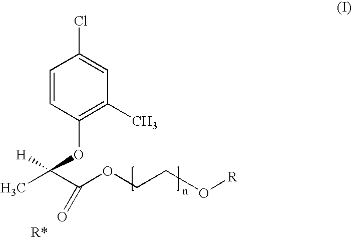
where
R represents H or a radical of the formula

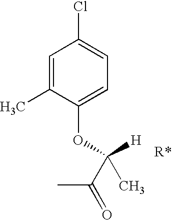

and

n represents an integer between 1 and 20 and

R* denotes the R configuration of the chiral carbon atom.

For the purposes of the present invention, enantiomerically enriched means enantiomerically pure compounds or mixtures of enantiomers of a compound in which one enantiomer is present in an enantiomeric excess, hereinbelow also referred to as ee, in comparison with the other enantiomer. Preferably, this enantiomeric excess amounts to 70 to 100% ee, especially preferably to 80% to 100 mol % and very especially preferably to 90 to 100 mol %.

For the purposes of the invention, enantiomerically enriched refers to the configuration of the carbon atom marked R* in formulae (I) and (III).

The present invention furthermore relates to the use of enantiomerically enriched compounds of the formula (I) or their mixtures with one another for the protection of industrial materials, in particular buildings, building materials and building auxiliaries, against root penetration thereinto and therethrough.

By employing the enantiomerically enriched polyethylene glycol esters according to the invention of R-(+)-2-(4-chloro-2-methylphenoxy)propionic acid and of the mixtures according to the invention, the amount required for preventing through-penetration of roots can be reduced markedly. This is particularly recommended for ecological reasons since only the active enantiomer is employed and inactive compounds are not introduced into the environment.

While it is known from crop protection that the herbicidal activity of R-(+)-2-(4-chloro-2-methylphenoxy)propionic acid in the free state is higher than that of the racemic 2-(4-chloro-2-methylphenoxy)propionic acid, the application of the herbicidal effects in crop protection to the root-penetration-inhibitory effects for example in films or roofing membranes is not obvious. In the case of herbicidal application, undesired plants are destroyed, while the use of root penetration inhibitors only intends to prevent the invasive root growth. Root penetration inhibitors, when employed in sealants, films or roofing membranes, show no herbicidal effect whatsoever, so that they are suitable for example as support and moisture barrier for vegetated roofs.

Furthermore, it must be considered as surprising that, in contrast to general technical knowledge, the compounds of the formula (I) according to the invention do not entail racemization, both under the production conditions and under the conditions of their incorporation into industrial materials, where temperatures of up to 200° C. are required.

Preferred enantiomerically enriched compounds of the general formula (I) or their mixtures with one another are those in which R has the abovementioned meaning and n represents an integer between 2 and 10.

Especially preferred enantiomerically enriched compounds of the general formula (I) or their mixtures with one another are those in which R has the abovementioned meaning and n represents an integer between 2 and 8.

Furthermore, the invention relates to any mixtures of enantiomerically enriched compounds of the general formula (I).

In general, these mixtures according to the invention comprise individual components of the general formula (I) in concentrations of in each case up to 80% by weight, the total of the individual components of the formula (I) adding up to 100% by weight. Preferred mixtures according to the invention are those which comprise the individual components of the general formula (I) where n=3 to 10 at in each case 0 to 80% by weight and the individual components of the general formula (I) where n=1 and the individual components of the general formula (I) where n=2 at in each case 0 to 5% by weight, the total of the individual components of the formula (I) adding up to 100% by weight.

Especially preferred mixtures according to the invention of compounds of the general formula (I) are those which comprise the individual components of the general formula (I) where n=3 to 8 and in each case up to 80% by weight, individual components of the general formula (I) where n=9 and the individual components of the general formula (I) where n=10 at in each case 0 to 10% by weight and the individual components of the general formula (I) where n=1 and the individual components of the general formula (I) where n=2 at in each case <1% by weight, the total of the individual components of the general formula (I) adding up to 100% by weight.

The enantiomerically enriched polyethylene glycol esters according to the invention of R-(+)-2-(4-chloro-2-methylphenoxy)propionic acid of the general formula (I) can be prepared by heating polyethylene glycol of the general formula (II), 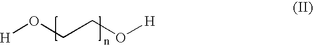
where
n represents an integer between 1 and 20, preferably an integer between 2 and 10 and very especially preferably an integer between 2 and 8 together with enantiomerically enriched R-(+)-2-(4-chloro-2-methylphenoxy)-propionic acid of the formula (III) 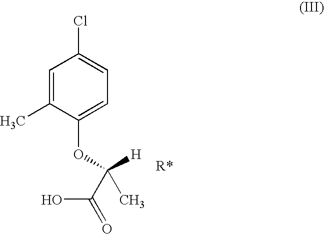
and distilling off the water of reaction which forms. If appropriate, the removal of the water of reaction by distillation can be supported by applying a vacuum. Moreover, catalysts such as, for example, acids, may also be employed, if appropriate.

The molar ratio between R-(+)-2-(4-chloro-2-methylphenoxy)propionic acid of the formula (III) and the polyethylene glycol of the formula (II) is generally between 1:1.2 and 2.2:1.

Preferably, the molar ratio between R-(+)-2-(4-chloro-2-methylphenoxy)propionic acid and polyethylene glycol of the formula (II) is between 1:1 and 2.1:1.

The reaction temperatures can be varied within a wide range. In general, they are between 100 and 250° C.

Preferably, the reaction temperatures are between 130 and 230° C., especially preferably between 150 and 210° C.

The removal of water of reaction by distillation can be supported by applying a vacuum. In this context, it is possible to apply a vacuum as early as at the beginning of the reaction or only during the course of the reaction.

In general, the vacuum can be lowered down to 0.5 mbar, a vacuum of up to 1 mbar preferably being employed.

The enantiomerically enriched R-(+)-2-(4-chloro-2-methylphenoxy)propionic acid employed in the reaction is known and commercially available as are the polyethylene glycols.

The mixtures of enantiomerically enriched compounds of the general formula (I) can either be obtained by mixing the enantiomerically enriched individual components of the general formula (I) or by employing mixtures of polyethylene glycols of the general formula (II) instead of pure polyethylene glycols in the preparation.

In general, the polyethylene glycol mixtures employed for the synthesis of the mixtures may comprise the individual polyethylene glycols of the general formula (II) in concentrations of in each case up to 80% by weight, the total of the individual components adding up to 100% by weight.

It is preferred to employ polyethylene glycol mixtures which comprise the individual polyethylene glycols of the general formula (II) where n=3 to 10 at in each case up to 80% by weight and the individual polyethylene glycols of the general formula (II) where n=1 and those of the formula (II) where n=2 at in each case 0 to 5% by weight, the total of the polyethylene glycols of the general formula (II) employed adding up to 100% by weight.

It is especially preferred to employ mixtures of polyethylene glycols of the general formula (II) which comprise the individual polyethylene glycols where n=3 to 8 and in each case up to 80% by weight, the polyethylene glycols of the general formula (II) where n=9 and the individual polyethylene glycols of the general formula (II) where n=10 at in each case 0 to 10% by weight and the individual polyethylene glycols of the general formula (II) where n=2 at in each case <1% by weight, the total of the polyethylene glycols of the formula (II) employed adding up to 100% by weight.

The enantiomerically enriched R-(+)-2-(4-chloro-2-methylphenoxy)propionic acid polyethylene glycol esters of the general formula (I) according to the invention and mixtures of these enantiomerically enriched R-(+)-2-(4-chloro-2-methylphenoxy)propionic acid polyethylene glycol esters of the formula (I) with one another can be used in particular for the protection of buildings such as sewers, pipes, houses, roofs, streets, bridge constructions, hydraulic structures and building materials and building adjuvants such as, for example, cement, polymer films, roofing membranes and products for the construction sector such as, for example, paints, sealants and insulants against root penetration thereinto and therethrough.

In general, the compounds of the formula (I) according to the invention or their mixtures can be applied directly to the industrial materials to be protected or mixed with these materials. Thus, for example, buildings can be treated directly with the compounds of the formula (I) according to the invention or their mixtures, or else they can be treated with products for the construction sector, for example those based on bitumen or tar pitches, which comprise one or more compounds of the formula (I) according to the invention. For example, the building materials and building adjuvants can be mixed with the compounds of the formula (I) according to the invention or their mixtures.

The products for the construction sector can be prepared by treating or mixing a commercially available product for the construction sector, for example based on bitumen or tar pitches or a paint or a sealant or insulating compound, with one or more compounds of the formula (I) according to the invention. The building materials, building auxiliaries and products for the construction sector generally comprise the enantiomerically enriched active ingredient of the formula (I) in an amount of from 0.01 to 20% by weight, preferably 0.01 to 5% by weight, especially 0.1 to 2% by weight, based on the respective preparation or product for the construction sector.

### EXAMPLES

### Example 1

### (Diesters of tetraethylene glycol with R-(+)-2-(4-chloro-2-methylphenoxy)propionic Acid)

60 g (0.28 mol) of R-(+)-2-(4-chloro-2-methylphenoxy)propionic acid, 24.7 g (0.13 mol) of tetraethylene glycol and 0.5 g of p-toluenesulfonic acid are combined in a round-bottomed flask equipped with distillation bridge and stirred for 6 hours at an internal temperature of 180° C. The water of reaction which formed distills off during this process. Thereafter, the temperature was reduced to 160° C., and further water of reaction distilled off under pressure of 80 mbar in the course of 6 hours. The residue was filtered through silica gel (ethyl acetate/toluene=1:1). Evaporation of the solvent gave 71 g (68% of theory) of the diester of tetraethylene glycol with R-(+)-2-(4-chloro-2-methylphenoxy)propionic acid as a pale red oil. [α]_{D}²=+15.4° (c=1, ethanol) with an enantiomeric excess of >95 mol %.

### Example 2

### (Diesters of tetraethylene glycol with rac-2-(4-chloro-2-methylphenoxy)propionic Acid/Comparative Compound)

60 g (0.28 mol) of rac-2-(4-chloro-2-methylphenoxy)propionic acid, 24.7 g (0.13 mol) of tetraethylene glycol and 0.5 g of p-toluenesulfonic acid are combined in a round-bottomed flask equipped with distillation bridge and stirred for 6 hours at an internal temperature of 180° C. The water of reaction which formed distills off during this process. Thereafter, the temperature was reduced to 160° C., and further water of reaction distilled off under pressure of 80 mbar in the course of 6 hours. The residue was filtered through silica gel (ethyl acetate/toluene=1:2). Evaporation of the solvent gave 82 g of the diester of tetraethylene glycol with rac-2-(4-chloro-2-methylphenoxy)propionic acid as an oil.

[α]_{D}²⁰=+0.1° (c=1, ethanol).

### Example 3

719 g (2.18 mol) of R-(+)-2-(4-chloro-2-methylphenoxy)propionic acid and 385 g of a glycol mixture consisting of 33.6% triethylene glycol, 65.5% tetraethylene glycol and 1.2% of pentaethylene glycol were introduced into a plane-ground reaction vessel equipped with distillation bridge, and the temperature was raised to 200° C. under atmospheric pressure within 4 hours. Water of reaction which formed together with some impurities distills off during this process. Thereafter, a vacuum (2 mbar) was applied to remove further water of reaction, and stirring was continued for 16 hours at 200° C. Thereafter, the mixture was cooled and the reaction product was drained via the bottom valve. This gave 1010 g of a brown oil.

To determine the optical purity, 1.5 g of NaOH were dissolved in 10 ml of ethanol, treated with 10 g of the above product and the mixture was stirred for 2 hours at room temperature. Thereafter, the mixture was poured into 100 ml of water, washed twice with CH₂Cl₂ and acidified with dilute HCl to pH=1-2. The oily product was then redissolved in 10% strength NaOH, washed with toluene, and the acid was reprecipitated by acidification with HCl (10% strength). After drying in vacuo, the optical rotation was determined: [α]_{D}²⁰=+15.3° (c=1, CHCl₃). The R-(+)-2-(4-chloro-2-methylphenoxy)propionic acid employed for the reaction had an optical rotation of [α]_{D}²⁰=+15.1° (c=1, CHCl₃). The resulting product had an enantiomeric excess of >95 mol %.

### Example 4

### (Comparative Compound of S-(−)-2-(4-chloro-2-methylphenoxy)propionic Acid)

39 g (0.18 mol) of S-(−)-2-(4-chloro-2-methylphenoxy)propionic acid, 16.41 g (0.08 mol) of tetraethylene glycol and 0.1 g of p-toluenesulfonic acid were combined in a round-bottomed flask equipped with distillation bridge and stirred for 6 hours at an internal temperature of 180° C. The water of reaction which formed distills off during this process. Thereafter, the temperature was reduced to 160° C., and further water of reaction distilled off under pressure of 80 mbar in the course of 6 hours. The residue was filtered through silica gel (ethyl acetate/toluene=1:1). Evaporation of the solvent gave 37.4 g (=75% of theory) of the diester of tetraethylene glycol with S-(−)-2-(4-chloro-2-methylphenoxy)propionic acid as orange oil.

[α]_{D}²⁰=−15.7° (c=1, ethanol).

### Example 5

(Tempering of the Product of Example 3)

4 g of the product of Example 3 were stirred for 48 hours at 180° C. in 40 ml of o-dichlorobenzene. Most of the o-dichlorobenzene was distilled off in vacuo. Thereafter, a mixture of 0.6 g of 50% strength NaOH and 4 ml of ethanol was added with cooling, and the mixture was stirred for 16 hours at room temperature. This was then poured into 40 ml of water and the pH value was brought to >10. The mixture was washed rapidly three times with CH₂Cl₂ and the aqueous phase was acidified with HCl, which resulted in the separation of an oil which crystallized slowly. This was filtered off with suction, dried in vacuo, stirred once again with n-hexane and again dried in vacuo. This gave 1.52 g of brown-beige crystals.

[α]_{D}²⁰=+15.3° (c=1, ethanol).

### Example 6

(Tempering of the Product of Example 3 with Additives)

4 g of the product of Example 3, 2 g of ground lime stone, 2 g of SBS rubber and 40 g of o-dichlorobenzene were stirred for 48 hours at 180° C.

First, 50 ml of methanol were added, insoluble material was filtered off and the filtrate was evaporated in vacuo. Thereafter, a mixture of 0.6 g of 50% strength NaOH and 4 ml of ethanol was added to the residue with cooling and the mixture was stirred for 16 hours at room temperature. This was then poured into 40 ml of water and the pH value was brought to >10. The mixture was washed rapidly three times with CH₂Cl₂ and the aqueous phase was acidified with HCl, which resulted in the separation of an oil which crystallized slowly. This was filtered off with suction, dried in vacuo, stirred once again with n-hexane and again dried in vacuo. This gave 1.55 g of beige crystals.

[α]_{D}²⁰=+15.6° (c=1, ethanol).

### Example 7

The compounds and mixtures of Examples 1 and 2 were tested for their activity for root penetration inhibition as specified in DIN 4062 (lupin test):

| | | Root penetration | Root penetration |
|---|---|---|---|
| Component | Concentration | into (number) | through (number) |
| Example 1 | 0.1% | approx. 40 | 0 |
| | 0.2% | approx. 15 | 0 |
| | 0.5% | 1 | 0 |
| Example 2 | 0.1% | approx. 40 | 1 |
| | 0.2% | approx. 40 | 0 |
| | 0.5% | approx. 10 | 0 |
| | 1.0% | 0 | 0 |
| Example 4 | 0.1% | >50 | >50 |
| (comparative | 0.2% | >50 | >50 |
| compound) | 0.5% | >50 | >50 |
| | 1.0% | approx. 40 | approx. 40 |

We claim:

1. Enantiomerically enriched R-(+)-2-(4-chloro-2-methylphenoxy)propionic acid polyethylene glycol esters of the general formula (I) and any of their mixtures with one another, 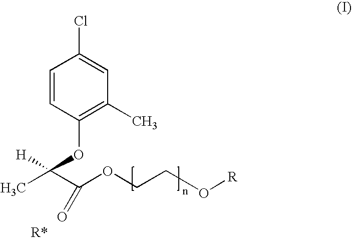
where
R represents H or a radical of the formula 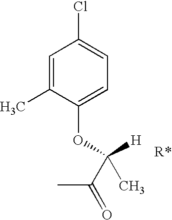
and
n represents an integer between 1 and 20 and
R* denotes the R configuration of the chiral carbon atom.

2. Enantiomerically enriched compounds as claimed in claim 1 or their mixtures with one another, wherein, in formula (I), n is an integer between 2 and 10.

3. A mixture comprising 0 to 80% by weight of at least two enantiomerically enriched compounds of the formula (I) as claimed in claim 1, wherein the total of the individual components of the formula (I) adds up to 100% by weight.

4. A method for the preparation of an enantiomerically enriched R-(+)-2-(4-chloro-2-methylphenoxy)propionic acid polyethylene glycol ester of the general formula (I) or mixtures thereof with one another as claimed in claim 1, characterized in that at least one polyethylene glycol of the general formula (II) 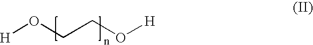
where
n represents a number between 1 and 20
is heated together with enantiomerically enriched R-(+)-2-(4-chloro-2-methylphenoxy)propionic acid of the formula 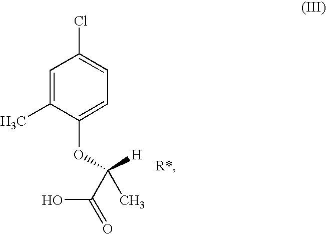
if appropriate in the presence of one or more catalysts and the water of reaction formed is distilled off.

5. The use of enantiomerically enriched polyethylene glycol esters of R-(+)-2-(4-chloro-2-methylphenoxy)propionic acid of the general formula (I) as claimed in at least one of claims 1 to 3 for the protection of industrial materials against root penetration thereinto and therethrough.

6. The use as claimed in claim 5, wherein the industrial materials are buildings, building materials and building auxiliaries.

7. A composition comprising enantiomerically enriched compounds of the formula (I) as claimed in at least one of claims 1 to 3 and at least one solvent or diluent and, if appropriate, further processing auxiliaries, fillers and additives.

8. A method for the protection of industrial materials against root penetration thereinto and therethrough, which comprises enantiomerically enriched compounds of the formula (I) as claimed in at least one of claims 1 to 3 being either applied directly to the industrial material to be protected, or mixed therewith, or the industrial material being treated with a composition as claimed in claim 7.

9. An industrial material comprising enantiomerically enriched compound of the formula (I) as claimed in at least one of claims 1 to 3.

10. The use of a composition as claimed in claim 7 for the protection of industrial materials against root penetration thereinto and therethrough.
